(19) United States
(12) Patent Application Publication
(10) Pub. No.: US 2018/0134638 A1
(43) Pub. Date: May 17, 2018

(54) IMPROVED PROCESS FOR SEPARATION OF ALKENES FROM ALKANES

(71) Applicant: COMPACT MEMBRANE SYSTEMS, INC., NEWPORT, DE (US)

(72) Inventor: YOSUKE KOIZUMI, BEAR, DE (US)

(21) Appl. No.: 15/573,222

(22) PCT Filed: May 6, 2016
(86) PCT No.: PCT/US16/31122

§ 371 Date: Nov. 10, 2017

Related U.S. Application Data
(60) Provisional application No. 62/159,668, filed on May 11, 2015.
(60) Provisional application No. 62/262,169, filed on Dec. 2, 2015.

(51) Int. Cl. C07C 7/144; B01D 53/22; B01D 71/32; B01D 71/82; C07C 7/00

(52) U.S. Cl. CPC C07C 7/144; B01D 53/228; C07C 7/005; B01D 71/82; B01D 71/32

(57) ABSTRACT

Alkenes may be separated from alkanes using membranes of Group 11 metal ionomers of sulfonic acid group contaning polymers, and the useful lifetime of these membranes is increased by passing the mixture containing the alkanes and alkenes over and/or through a material which reduces H₂S concentrations in the feed stream before exposing the membrane to the feed stream.

### GOVERNMENT RIGHTS

Support was provided under Department of Energy awards of DE-500004672 and DE-500007510. The U.S. government has rights in this patent application.

### FIELD OF THE INVENTION

Membranes containing Group 11 metal ionomers, and used for the separation of alkenes from alkanes, have longer lifetimes if the feed gas mixture of alkenes(s) and alkane(s) is passed over or through a material that reduces the concentration of H₂S in the gases.

### TECHNICAL BACKGROUND

Nonporous, but permeable, membranes have been used to separate various types of chemicals for a long time. For instance certain types of semipermeable membranes are used to separate water from seawater, or oxygen from nitrogen, or alkenes from alkenes.

The separation of alkenes from alkenes can be accomplished using a silver ionomer of a fluorinated polymer. Perhaps because fluoropolymers are more stable to oxidation than unfluorinated polymers, the silver ionomers of fluorinated polymers are often more stable than unfluorinated polymers. Also polymers which contain fluoro substituents near, for instance sulfonic acid or carboxyl groups, tend to be very strong acids (sometimes called “super acids), and the silver salts may be more stable.

In oil refineries or alkene polymerization plants sometimes one has mixtures of alkenes and alkanes and one desires to separate the alkenes from the alkanes. This may be relatively easy if these two types of compounds have significant differences in boiling points, but separation of such compounds with similar boiling points are more difficult and expensive, especially if the boiling points are lower in temperature. For instance propane boils at −44.5° C. and propylene boils at −47.8° C. Separation of these two compounds by cryogenic distillation is very expensive because of high equipment and energy costs. Therefore cheaper, less energy intensive methods of separation are desirable.

Use of ionomeric membranes is one such method, but it has been found that such ionomeric membranes often lose activity with time of use, i.e., the permeability of the ionomer decreases with time even using mixtures of alkenes and alkanes that are relatively pure, and/or the selectivity of membrane decreases. Therefore methods of increasing the useful lifetime of such membranes are desired.

Another method of separating alkenes from alkanes is to use a membrane which is a simple mixture of an “inert” polymer and a silver salt such as AgBF₄ or AgNO₃, The polymer used basically acts as binder for the silver salt in the membrane layer, but these types of membranes are known to be degraded when exposed to light, and/or certain chemical poisons such as H₂S, acetylene, and hydrogen.

U.S. Pat. No. 5,191,151 to Erikson et al. describes the separation of lower alkenes (containing 2 to 4 carbon atoms) from lower alkanes (containing one to six carbon atoms) using a membrane which is a silver ionomer of a polymer of tetrafluoroethylene (TFE) and a perfluorovinyl ether containing a terminal precursor group to a sulfonic acid. U.S. Patent Application 2015/0025293 to Feiring et al. describes the use of a membrane which is a silver ionomer of a fluorinated polymer. Neither of these patents describe pretreating the alkene-alkane mixture to improve membrane lifetime.

T. C. Merkel, et al., Journal of Membrane Science, vol. 447 (2013), pp. 177-189 report that membranes that contain mixtures of silver salts such AgBF₄ simply dispersed in a polyether block amide can be used for the separation of alkenes from alkanes, but is rapidly “poisoned” by a variety types of materials including H₂S. The authors do not use, test or comment on ionomers..

### SUMMARY OF THE INVENTION

Described herein is a process for separating one or more alkanes from one or more alkenes, comprising:
(a) providing a membrane comprising a layer of an ionomer, said ionomer comprising repeat units derived from one or more fluorinated monomers and a repeat unit having a Group 11 metal salt of a sulfonic acid group, said membrane having a first side and a second side, and providing that carbon-fluorine groups are at least 30% of the total of said carbon-fluorine groups and carbon-hydrogen groups present in said ionomer;
(b) exposing said first side to a feed composition comprising a mixture of one or more alkanes and one or more alkenes;
(c) providing a driving force; and
(d) producing a second mixture, on a second side of said membrane, having a higher ratio of alkene to alkane than said first mixture;

and wherein the improvement comprises said feed composition is passed through and/or over a material which reduces the concentration of H₂S in said mixture.

### DESCRIPTION OF THE FIGURES

FIG. 1 shows the comparative effect with time of the presence of 0.8 ppm by volume of H₂S present in an alkane/alkene feed stream on the permeance and selectivity of a silver ionomer containing membrane, with and without scrubbing the feed stream with a CuSO₄ solution.

FIG. 2 shows the comparative effect with time of the presence of 0.8 ppm by volume of H₂S present in an alkane/alkene feed stream on the permeance and selectivity of a silver ionomer containing membrane derived from Nafion®, with and without scrubbing the feed stream with a CuSO₄ solution.

### DETAILS OF THE INVENTION

Herein certain terms are used, and some of them are defined below:

Of the total of the carbon-hydrogen groups and the carbon fluorine groups in the ionomer, 30% or more are carbon-fluorine groups, preferably 40% or more, more preferably 60% or more, very preferably 80% or more are carbon fluorine groups. By a carbon-hydrogen group is meant a hydrogen atom bound directly to a carbon atom, while a carbon-fluorine group is a fluorine atom bound directly to a carbon atom. Thus —CF₂— groups contains 2 carbon fluorine groups, while a —CH₃ group contains 3 carbon-hydrogen groups. Thus in a homopolymer of vinylidene fluoride, in which the repeat groups are —CH₂CF₂— the carbon-hydrogen groups and the carbon fluorine groups are each 50% of the total of carbon-hydrogen plus carbon-fluorine groups present. In a copolymer of 20 mole percent CF₂═CFOCF₂CF(CF₃)OCF₂CF₂SO₂F and 80 mole percent vinylidene fluoride the carbon-hydrogen groups are 27.6% of the total of the carbon-fluorine plus carbon hydrogen groups present. The relative amount of carbon-fluorine and carbon hydrogen groups present can be determined by NMR Spectroscopy, for instance using ¹⁴C NMR, or a combination of ¹⁹F and ¹H spectroscopy.

By a “driving force” in the separation of the alkene and alkane in the gaseous state is generally meant that the partial pressure of alkene on the first (“feed”) side of the membrane is higher than the partial pressure of alkene on the second (“product”) side of the membrane. For instance this may be accomplished by several methods or a combination thereof. One is pressurizing first side to increase the partial pressure of alkene on the first side, second is sweeping the second side by inert gas such as nitrogen to lower the partial pressure of the alkene on the second side, and third is reducing pressure of second side by vacuum pump to lower the partial pressure of the alkene on the second side. These and other known methods in the art of applying a driving force may be used.

This may be quantified for a separation of gases to some extent by a mathematical relationship:

QₐαFₐ(P1ₐ-P2ₐ)

wherein Qₐ is the flow rate of component “a” through the membrane, Fₐ is the permeance of component a through the membrane, P1ₐ is the partial pressure on the first (feed) side, and P2ₐ is the partial pressure on the second (product) side.

By a membrane containing a Group 11 metal ionomer is meant a membrane comprising a thin nonporous layer of the metal ionomer and one or more other polymeric layers which physically support or reinforce the Group 11 metal ionomer layer. Preferably the Group 11 metal ionomer layer is about 0.1 μm to about 1.0 μm thick, more preferably about 0.2 μm to about 0.5 μm thick. The other layer(s) should preferably be relatively permeable to the alkenes and alkanes to be separated, and not themselves have much if any tendency to separate alkenes and alkanes.

By a water insoluble metal sulfide is meant a metal sulfide whose Solubility Product is less than about 1×10⁻¹⁰ in water at 25° C.

In one preferred embodiment of the invention the Group 11 metal is copper or silver, more preferably silver.

In the Group 11 metal ionomer, and its precursor acidic form, the repeat units that contain the pendant sulfonic acid (or readily converted to sulfonic acid) groups are preferably at least about 5 mole percent of the total repeat units present, more preferably at least about 10 mole percent, very preferably at least about 15 mole percent, and especially preferably at least about 22 mole percent. It is preferred that the repeat units that contain the pendant acid groups are no more than 45 mole percent of the repeat units present in the silver ionomer or its precursor acid form. It is to be understood that any minimum amount of such repeat units and any maximum amount of such repeat units may be combined to form a preferred range of the amount of these repeat units,

Useful monomers containing a sulfonic acid group or a precursor to a sulfonic acid group include one or more of CF₂═CFOCF₂CF₂SO₂F and CF₂═CFOCF₂CF(CF₃)OCF₂CF₂SO₂F, and CF₂═CFOCF₂CF(CF₃)OCF₂CF₂SO₂F is preferred. Precursors to sulfonate salts include sulfonyl halides, sulfonyl esters, and sulfonyl amides, all of which may be hydrolyzed after polymer formation to the corresponding sulfonic acid. The polymeric portion of the ionomers may contain repeat units derived from other monomers, as long as other compositional limits of the polymer, such as the minimum fraction of carbon-fluorine groups present. Useful monomers include one or more of tetrafluoroethylene, chlorotrifluoroethylene, vinyl fluoride, trifluororethylene, vinylidene fluoride, and ethylene.

Commercially available fluorinated polymers which contain pendant sulfonic acid groups (or their precursors) include Nation® membranes and dispersion available from The Chemours Co., Wilmington, Del. 19899, U.S.A. These membranes and dispersions are believed to be copolymers of TFE and a perfluorovinyl ether containing a terminal precursor group to a sulfonic acid. Another suitable type of fluoropolymer contains pendant sulfonic acid groups is Aquivion® PFSA available from Solvay SA, 1120 Brussels, Belgium. The Aquivion® polymers are made by polymerizing TFE and F₂C═CF—O—CF₂—CF₂—SO₂F and are available as, or readily converted to the sulfonic acid form.

Another type of useful monomer is a perfluorinated cyclic or cyclizable monomer. By a cyclic perfluorinated monomer is meant a perfluorinated alkene wherein a double bond of the alkene is in the ring or the double bond is an exo double bond wherein one end of the double bond is at a ring carbon atom. By a cyclizable perfluorinated monomer is meant a noncyclic perfluorinated compound containing two alkeneic bonds, and that on polymerization forms a cyclic structure in the main chain of the polymer (see for instance N. Sugiyama, Perfluoropolymers Obtained by Cyclopolymereization and Their Applications, in J. Schiers, Ed., Modern Fluoropolymers. John Wiley & Sons, N.Y., 1997, p. 541-555, which is hereby included by reference). Such perfluorinated cyclic and cyclizable compounds include perfluoro(2,2-dimethyl-1,3-dioxole), perfluoro(2-methylene-4-methyl-1,3-dioxolane), a perfluoroalkenyl perfluorovinyl ether, and 2,2,4-trifluoro-5-trifluoroimethoxy-1,3-dioxole.

The ionomers may be produced by methods described in this application, US Patent Application 2015/0025293 to Feiring et al,. and U.S. Pat. No. 5,191,151 to Erikson et al. and which are both hereby included by reference. These references also describe syntheses of these ionomers.

In the separations using these membranes the feed and/or product streams may be gases and/or liquids. In one preferred embodiment the feed stream mixture of alkenes and alkenes is gaseous, and the product stream, after enrichment in the alkenes is also gaseous. It is preferred if at least one of ethylene, propylene, 1-butene and 2-butenes present in the alkene/akane mixture where the concentration of alkene with be increased.

When carrying out the process of separation of alkenes from alkanes using Group 11 metal ionomers, especially silver ionomers, as described herein, it is preferred that the feed alkane/alkene mixture (that is before separation) be passed through and/or over a material that will reduce the concentration of sulfur compounds, such as H₂S, from the mixture. This is true whether or not that membrane has been exposed to and/or contains a strong Bronsted acid. Preferred polymers, and ionomers derived from them, are as described herein.

Although not wishing to be bound by theory in this instance, it is possible that that the feed gas mixture of alkenes and alkanes used in this process may contain trace amounts of H₂S and perhaps other reduced sulfur compounds such as thiols, These may be responsible for the gradual “poisoning” of the membrane and loss of permeability to alkenes. It has been found that contacting the feed gas mixture with compositions that reduce the concentration of sulfur compounds in gas and/or liquid mixtures extends the useful lifetimes of these ionomer membranes.

The removal of sulfur compounds such as H₂S and sometimes other reduced sulfide compounds such as thiols, organic sulfides, and similar compounds is a well-known technology, see for instance A. L. Kohl, Gas Purification, 5^{th} Ed., Gulf Publishing Co, Houston, Tex., Among these methods are passing the gas through a solution containing certain metal ions [see H. ter Maat, et. al., Separation and Purification Technology, vol. 43, p. 183-197 (2005) and U.S. Pat. No. 7,067,093], and passage over certain metal oxides (see U.S. Pat. No. 7,067,093), All of the references cited in this paragraph are hereby included by reference. One of the reasons for the development of this technology is the fact that H₂S is present in many naturally occurring fuel sources such as natural gas and crude oil. In oil refineries, partially because there are sulfur containing compounds in crude oil, H₂S is often generated in the preparation and purification of many products. H₂S is toxic, explosive (in higher concentrations), causes serious air pollution problems, and as is well known has a very noxious smell. In some instances it can also poison catalysts used in various chemical processes. For these and other reasons removal of H₂S and similar compounds from refinery streams natural gas, and other materials is highly desirable.

Any of the processes and/or materials developed for the removal sulfur compounds from process streams, especially gaseous process streams, may be used herein to extend the useful lifetime of the ionomer containing membrane. Preferably whatever process and/or material is used for the removal should be very efficient so any H₂S present will reduced to very low levels. Also, such process and/or material should preferably not react significantly with any of compounds in the feed stream. There are many materials sold commercially designed to reduce the concentration of H₂S in the compositions, and some of them are SULFATREAT® and SELECT HC® supplied by M-I Swaco, Sclumberger Technology Corp., 300 Schlumberger Dr., Sugar Land, Tex. 77478, U.S.A., and H2CPlus® System and others from MV Technologies, 751 Pine Ridge Rd., Golden, Colo. 80403, U.S.A..

A preferred method of purification of the feed gas mixture is passing the feed gas through an aqueous solution of certain metal cations such as Fe(II), Zn(II), Ag(I), Ni(II), Sn(II) and Cu(II). Generally speaking these metal cations form water insoluble sulfides. Details of such a process may be found in H. ter Maat, previously cited. In one preferred embodiment, the metal cation is Cu(II), for instance as CuSO₄. Not only does this reduce the concentration of H₂S and other sulfur compounds in the feed gas, but it also humidifies the feed gas mixture, which is preferred.

Preferably the total sulfur content of the feed mixture should be reduced by about at least 50%, more preferably about at least 80%,. more preferably about at least 90%, very preferably about at least 95%, and especially preferably about at least 99.0% after treatment to reduce sulfur compound concentrations. The percentage which must be removed to improve membrane lifetime will depend on the characteristics of the alkene-alkane mixture (for instance whether gaseous or liquid), the sulfur compounds present, and the characteristics of the membrane itself. The effective amount of removal of sulfur compounds in any particular circumstance can be readily determined by routine experimentation. Total sulfur contents, and specific sulfur compound such as H₂S contents may be determined by a large number of various ASTM tests, depending on the substance being tested. For instance ASTM D3246-15, Standard Test Method for Sulfur in Petroleum Gas by Oxidative Microcoulometry and ASTM D6667-14, Standard Test Method for Determination of Total Volatile Sulfur in Gaseous Hydrocarbons and Liquefied Petroleum Gases by Ultraviolet Fluorescence may be applicable to gaseous alkane-alkene feed mixtures before and after treatment to reduce sulfur compound concentrations, while ASTM5453-12, Standard Method for Determination of Total Sulfur in Light Hydrocarbons, Spark Ignition Engine Fuel, Diesel Engine Fuel, and Engine Oil by Ultraviolet Fluorescence, and ASTM D7039-15 may be applicable to liquid alkane-alkene feed mixtures before and after treatment to reduce sulfur compound concentrations.

Preferably after treatment with the sulfur compound removing material the alkene/alkane mixture (before contacting the separation membrane) will have less than about 500 parts per billion by weight, more preferably less than about 300 parts per billion by weight, of total sulfur in it, when the sulfur compounds are measured by ASTM Method D7493-14. This method may also be used to determine the percentage of the total sulfur reduction after treatment with the sulfur compound removing material.

In another preferred for, for gaseous alkane-alkene mixtures it is preferred that the total amount of sulfur present after treatment to reduce sulfur compound concentrations is less than about 0.01 ppm by volume and more preferably less than about 1 ppb by volume , when the total sulfur content present is assumed to be H₂S.

Determination of Permeance and Selectivity for Olefin/Alkane Separations

Except where otherwise noted, for determinations of permeance (GPU, reported in units of sec/cm²·s·cm Hg) and selectivity the following procedure was used. A 47 mm flat disc membrane was punched from a larger flat sheet 3 inch composite membrane. The 47 mm disc is then placed in a stainless steel cross flow testing cell comprised of a feed port, retentate port, a sweep inlet port, and a permeate port. Four hex bolts were used to tightly secure the membrane in the testing cell with a total active area of 13.85 cm².

The cell was placed in a testing apparatus comprising of a feed line, a retentate line, a sweep line, and a permeate line. The feed consisted of a mixture of an olefin (propylene) gas and a paraffin (propane) gas. Each gas was supplied from a separate cylinder. For olefin, polymer grade propylene (99.5 vol % purity) was used and for paraffin, 99.9 vol % purity propane was used. The two gases were then fed to their respective mass flow controllers where a mixture of any composition can be made. The standard mixing composition was 20 vol % olefin and 80 mol % paraffin at a total gas flow rate of 200 mL/min. The mixed gas was fed through a water bubbler to humidify the gas mixture bringing the relative humidity to greater than 90%. A back pressure regulator is used in the retentate line to control the feed pressure to the membrane. The feed pressure was normally kept at 60 psig (0.41 MPa) after the back pressure regulator the gas is vented.

The sweep line consisted of a pure humidified nitrogen stream. Nitrogen from a cylinder was connected to a mass flow controller. The mass flow controller was set to a flow of 300 mL/min. The nitrogen was fed to a water bubbler to bring the relative humidity to greater than 90%. After the bubbler the nitrogen was fed to the sweep port of the membrane to carry any permeating gas through to the permeate port.

The permeate line consisted of the permeated gas through the membrane and the sweep gas as well as water vapor. The permeate was connected to a three way valve so flow measurements could be taken. A Varian® 450 GC gas chromatograph (GC) with a GS-GasPro capillary column (0.32 mm, 30 m) was used to analyze the ratio of the olefin and paraffin in the permeate stream. The pressure in the permeate side was typically between 1.20 and 1.70 psig (8.3 to 11.7 kPa). Experiments were carried out at room temperature.

During experiment the following were recorded: feed pressure, permeate pressure, temperature, sweep-in flow rate (nitrogen+water vapor) and total permeate flow rate (permeate+nitrogen+water vapor).

From the results recorded the following were determined: all individual feed partial pressures based on feed flows and feed pressure; all individual permeate flows based on measured permeate flow, sweep flows, and composition from the GC; all individual permeate partial pressures based on permeate flows and permeate pressures. From these the transmembrane partial pressure difference of individual component were calculated. From the equation for permeance

Qᵢ=Fᵢ/(A.Δpᵢ)

wherein, Qᵢ=permeance of species ‘i’, Fᵢ=Permeate flow rate of species ‘i’Δpᵢ=transmembrane partial pressure difference of species ‘i’, and A is the area of the membrane (13.85 cm²), the permeance(Qᵢ) was calculated.

In the Examples certain abbreviations are used, and they are:

HFPO—hexafluoropropylene oxide (For preparation of HFPO dimer peroxide see U.S. Pat. No. 7,112,314, which is hereby included by reference. HFPO dimer [2062-98-8] is available from Synquest Laboratories, Alachua, Fl., U.S.A.)

PDD—perfluoro(2,2-dimethyl-1,3-dioxole)

SEFVE—CF₂═CFOCF₂CF(CF₃)OCF₂CF₂SO₂F

VF—vinyl fluoride (H₂C═CHF)

PAN—poly(acrylonitrile)

### EXAMPLE 1

### Synthesis of PDD/VF/SEFVE (Feed Ratio 100:200:150) Copolymer and Hydrolysis.

Into a 150 mL stainless steel pressure vessel, after argon purging for 5 minutes, were added a magnetic stirring bar, 3.66 g PDD, 10.04 g SEFVE, 15 mL of Vertrel ®XF (2,3-dihydrodecafluoropentane, available from the Chemours Company), 0.6 mL of HFPO dimer peroxide solution (0.12M), and then charged 1.38 g of vinyl fluoride gas at 0° C. The reaction mixture was sealed in the pressure vessel and stirred at room temperature in a water bath. After 5.5 hours of reaction, the reaction vessel was opened to ambient air, 10 mL acetone and 40 mL methanol was added to the reaction mixture. The resulting gel like precipitate was transferred to a glass dish and dried in oven at 100° C. overnight to yield 9.1 g PDD/VF/SEFVE terpolymer as a colorless solid (Tg 18° C.). Anal: Found: C, 24.92; H, 0.55; S, 5.01. Intrinsic viscosity: 0.389 dL/g. From the elemental analysis, the polymer composition was estimated as 21% PDD, 43% VF and 37% SEFVE.

Into a 250 mL round bottom flask, were added 5.8 g of the terpolymer synthesized in the previous paragraph, 20 mL deionized water, 80 mL of methanol, 2.0 g ammonium carbonate and a magnetic stirring bar. The reaction mixture was stirred and maintained at 50-60° C. After overnight reaction, a clear solution was obtained. 80 mL 2.0 M hydrochloric acid was added to the mixture and methanol in the mixture was evaporated under heating to form a gel like precipitate. The liquid was decanted and 50 mL of 2.0 M hydrochloric acid was added and stirred for 30 minutes. The liquid was decanted and 80 mL of deionized water was added and then stirred for 30 minutes. After the liquid decanting, the water washing was repeated twice and the solid residue was dried in a vacuum oven at 60° C. for 3 hours. A brownish solid (4.6 g) containing free sulfonic acid groups was obtained.

Obtained brownish solid (4.6 g) was placed in a glass bottle and 20 ml of 30% H₂O₂ in water was added, this mixture was stirred for overnight, filtered, washed with deionized water and dried under the vacuum.

### EXAMPLE 2

### Membrane Preparation

The polymer obtained by Example 1 (0.1 g) was dissolved in 5 g of ethanol and 20 mg of silver nitrate was added. After stirring for 2 hour, the solution was filtered through a glass filter having 1 μm of pore size.

A substrate was prepared by coating a 0.3 weight % solution of Teflon®AF2400 (available from Chemours Co, Wilmington, Del. 19898, U.S.A.) [for further information about Teflon®AF, see P. R. Resnick, et al., Teflon AF Amorphous Fluoropolymers, J. Science, Ed., Modern Fluoropolymers, John Wiley & Sons, N.Y., 1997, p. 397-420, which is hereby included by reference and reported to be 87 mol % 2,2bis(trifluoromethyl)-4,5-difluoro-1,3-dioxole and 13 mol % tetrafluoroethylene, I. Pinnau & L. G. Toy, Journal of Membrane Science, 109 (1996), pp. 125-133, which is hereby included by reference] in Fluorinert®770 (available from 3M Corp., 3M Center, Sty. Paul, Minn., U.S.A.) on a PAN350 membrane made by Nanostone Water, 10250 Valley View Rd., Eden Prairie, Minn. 53344, U.S.A.) (PAN350 is an ultrafiltration membrane made of polyacrylonitrile)

The coating above silver ionomer solution on the Teflon AF coated PAN350 membrane was done at <30% relative humidity. Two membranes were coated for further evaluation and punched 47 mm diameter of circular shape, named Membrane A and Membrane B.

### EXAMPLE 3

### Membrane Testing

Membranes A and B were set in filter cells, respectively. The first side of Membrane A was loaded with 60 psi of 0.8 ppm H₂S in propylene, and the second side was swept by 100 ml/min of nitrogen humidified through water bubbler. The first side of Membrane B was loaded with 60 psi of 0.8 ppm H₂S in propylene which was passed through a bubbler filled with 0.1M CuSO₄ aqueous solution, and the second side was swept by 100 ml/min of nitrogen humidified through water bubbler at ambient pressure. At periodic intervals each membrane was tested for propane-propylene permeance under the following conditions.

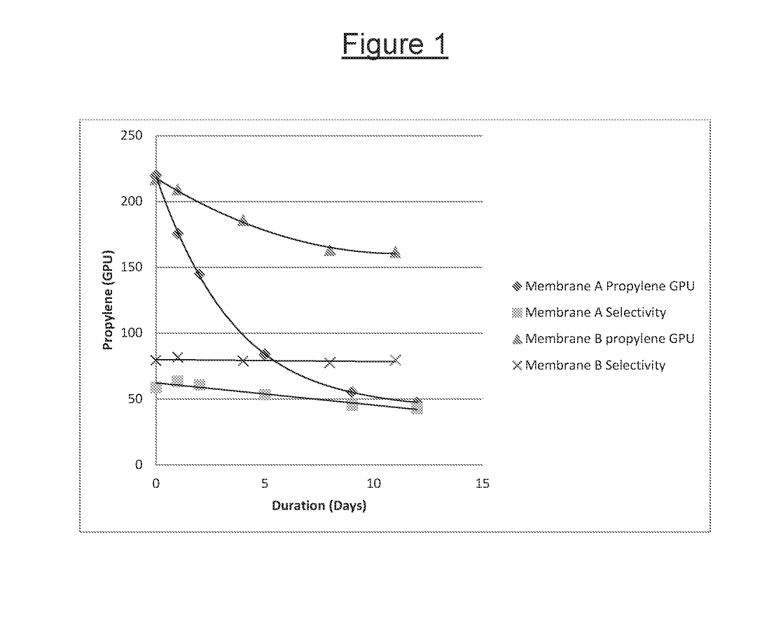

The feed gas composition, 20 mole % propylene (polymer synthesis grade), and 80 mole % of propane was humidified by passing it through a water bubbler. The total flow rate of both gases was 200 mL/min. The feed gas (mixture of propane and propylene) was 60 psi, and sweep gas on the second side of the membrane was humidified nitrogen at ambient pressure (<0.3 psig). The permeate from the second side of the membrane was analyzed by GC to determine the molar ratio of propane and propylene. Permeances (GPU) are given in cm³/cm²/sec/cm Hg×10⁵. Permeance and selectivity results are shown in FIG. 1.

### EXAMPLE 4

### Membrane Preparation from Nafion® Dispersion

Nafion® D2020 dispersion, 0.5 g, (obtained from DuPont Fuel Cells, P.O. Box 80701, Wilmington, Del., 19880-0701, U.S.A., and reportedly containing 20 weight percent polymer, about 34 wt % of water, and about 46 wt. % of 1-propanol, 1.03-1.12 meq/g of acid capacity on a polymer basis, and in the sulfonic acid form) was diluted to 2 wt. % of polymer concentration with ethanol, and then 19 mg of silver nitrate was added. After stirring the solution for 1-2 hours, the silver nitrate had dissolved. The solution was then filtered through a glass fiber filter having a nominal 1.2 um pore size. In a glove box this solution was coated onto Teflon® AF2400 coated onto PAN350 membrane at less than 20% relative humidity. The coated membrane was left at ambient temperature for 30 minutes, and put in an oven at 70° C. for 1 hour.

Two membranes were coated for further evaluation and punched 47 mm diameter of circular shape, named Membrane C and Membrane D.

### EXAMPLE 5

### Membrane Testing

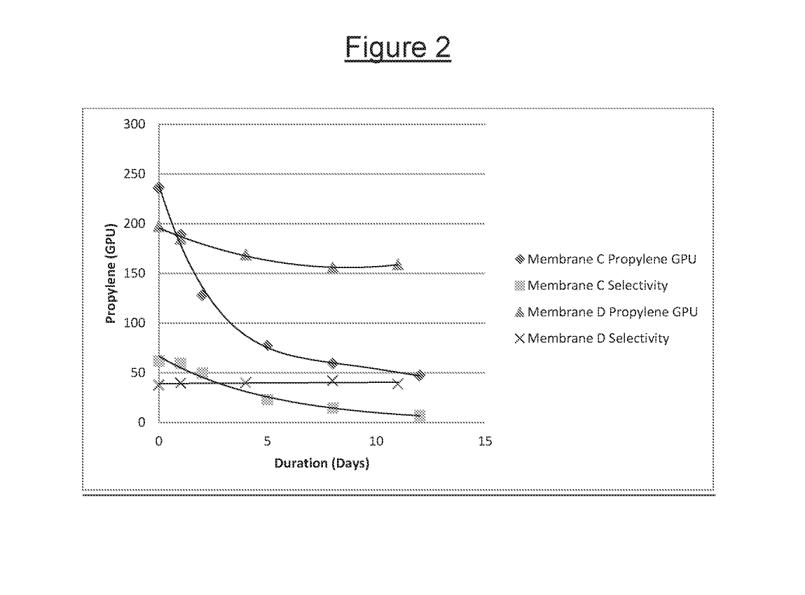

Membrane C and D were set in filter cells, respectively. The first side of Membrane C was loaded with 60 psi of 0.8 ppm H₂S in propylene, and the second side was swept by 100 ml/min of nitrogen humidified through water bubbler. The first side of Membrane D was loaded with 60 psi of 0.8 ppm H₂S in propylene which was passed through a bubbler filled with 0.1M CuSO₄ aqueous solution, and the second side was swept by 100 ml/min of nitrogen humidified through water bubbler at ambient pressure. At periodic intervals each membrane was tested for propane-propylene permeance under the following conditions described in example 3. Permeance and selectivity results are shown in FIG. 2.

1. A process for separating one or more alkanes from one or more alkenes, comprising:
(a) providing a membrane comprising a layer of an ionomer, said ionomer comprising repeat units derived from one or more fluorinated monomers and a repeat unit having a Group 11 metal salt of a sulfonic acid group, said membrane having a first side and a second side, providing that carbon-fluorine groups are at least 30% of the total of said carbon-fluorine groups and carbon-hydrogen groups present in said ionomer;
(b) exposing said first side to a feed composition comprising a gaseous first mixture of one or more alkanes and one or more alkenes;
(c) providing a driving force; and
(d) producing a second mixture, on a second side of said membrane, having a higher ratio of alkene to alkane than said first mixture;
and wherein the improvement comprises said feed composition is passed through and/or over a material which reduces the concentration of one or more sulfur containing compounds in said first mixture.

2. The process as recited in claim 1 wherein said Group 11 metal is silver.

3. The process as recited in claim 2 wherein the repeat units that contain said pendant sulfonic acid groups or a monomer containing a precursor of a sulfonic acid group at least about 10 mole percent of said repeat units present.

4. The process as recited in claim 2 wherein said carbon-fluorine groups are at least about 60% of the total of said carbon-fluorine and said carbon hydrogen present in said ionomer.

5. The process of claim 1 wherein said first mixture is a gas.

6. The process of claim 1 wherein said second mixture is a gas.

7. The process of claim 1 wherein said sulfur compound comprises H2S.

8. The process of claim 1 wherein said material comprises an aqueous solution containing a metal cation whose sulfide is insoluble in water.

9. The process as recited in claim 8 wherein said metal cation is Cu(II), Fe(II) or Zn(II).

10. The process as recited in claim 9 wherein said metal cation is Cu(II).

11. The process as recited in claim 10 wherein said Cu(II) is present as CuSO4.

12. The process of claim 1 wherein said alkenes comprise one or more of ethylene, propylene, 1-butene, and 2-butene.

13. The process as recited in claim 8 wherein said sulfur compounds comprises H2S.

14. The process of claim 2 wherein said first mixture is a gas.

15. The process of claim 3 wherein said first mixture is a gas.

16. The process of claim 4 wherein said first mixture is a gas.

17. The process of claim 2 wherein said second mixture is a gas.

18. The process of claim 3 wherein said second mixture is a gas.

19. The process of claim 4 wherein said second mixture is a gas.

20. The process of claim 2 wherein said sulfur compound comprises H2S.

21. The process of claim 3 wherein said sulfur compound comprises H2S.

22. The process of claim 4 wherein said sulfur compound comprises H2S.

23. The process of claim 2 wherein said material comprises an aqueous solution containing a metal cation whose sulfide is insoluble in water.

24. The process as recited in claim 23 wherein said metal cation is Cu(II), Fe(II) or Zn(II).

25. The process as recited in claim 24 wherein said metal cation is Cu(II).

26. The process as recited in claim 25 wherein said Cu(II) is present as CuSO4.

27. The process as recited in claim 23 wherein said sulfur compounds comprises H2S.

28. The process of claim 3 wherein said material comprises an aqueous solution containing a metal cation whose sulfide is insoluble in water.

29. The process as recited in claim 28 wherein said metal cation is Cu(II), Fe(II) or Zn(II).

30. The process as recited in claim 29 wherein said metal cation is Cu(II).

31. The process as recited in claim 30 wherein said Cu(II) is present as CuSO4.

32. The process as recited in claim 28 wherein said sulfur compounds comprises H2S.

33. The process of claim 4 wherein said material comprises an aqueous solution containing a metal cation whose sulfide is insoluble in water.

34. The process as recited in claim 33 wherein said metal cation is Cu(II), Fe(II) or Zn(II).

35. The process as recited in claim 34 wherein said metal cation is Cu(II).

36. The process as recited in claim 35 wherein said Cu(II) is present as CuSO4.

37. The process as recited in claim 33 wherein said sulfur compounds comprises H2S.

38. The process of claim 2 wherein said alkenes comprise one or more of ethylene, propylene, 1-butene, and 2-butene.

39. The process of claim 3 wherein said alkenes comprise one or more of ethylene, propylene, 1-butene, and 2-butene.

40. The process of claim 4 wherein said alkenes comprise one or more of ethylene, propylene, 1-butene, and 2-butene.
